SEQUENCE LISTING (18 sequences)

SEQ ID NO: 1 - DNA, 20 bp, synthetic construct
  source 1..20: mol_type = other DNA
cctctgcgctggaatcagtc

SEQ ID NO: 2 - DNA, 22 bp, synthetic construct
  source 1..22: mol_type = other DNA
ttctttctcgtcgcctcttttt

SEQ ID NO: 3 - DNA, 22 bp, synthetic construct
  source 1..22: mol_type = other DNA
gtatgagcccagagtcctatct

SEQ ID NO: 4 - DNA, 22 bp, synthetic construct
  source 1..22: mol_type = other DNA
cacattgacgctcctgactatc

SEQ ID NO: 5 - DNA, 19 bp, synthetic construct
  source 1..19: mol_type = other DNA
gaaccctggttacctgggc

SEQ ID NO: 6 - DNA, 22 bp, synthetic construct
  source 1..22: mol_type = other DNA
cgcagcaagtattcaaggttgt

SEQ ID NO: 7 - DNA, 21 bp, synthetic construct
  source 1..21: mol_type = other DNA
ggaaacagagtggtcattccc

SEQ ID NO: 8 - DNA, 21 bp, synthetic construct
  source 1..21: mol_type = other DNA
ctgcttgagccgttcattctc

SEQ ID NO: 9 - DNA, 19 bp, synthetic construct
  source 1..19: mol_type = other DNA
cgtcccgagtttgactggc

SEQ ID NO: 10 - DNA, 20 bp, synthetic construct
  source 1..20: mol_type = other DNA
gatgcgaggcatgagtctct

SEQ ID NO: 11 - DNA, 21 bp, synthetic construct
  source 1..21: mol_type = other DNA
catgtacgttgctatccaggc

SEQ ID NO: 12 - DNA, 21 bp, synthetic construct
  source 1..21: mol_type = other DNA
ctccttaatgtcacgcacgat

SEQ ID NO: 13 - DNA, 21 bp, synthetic construct
  source 1..21: mol_type = other DNA
  misc_feature 1..19: note = RNA
  misc_feature 20..21: note = DNA
ccaagacctggatcacgaatt

SEQ ID NO: 14 - DNA, 21 bp, synthetic construct
  source 1..21: mol_type = other DNA
  misc_feature 1..19: note = RNA
  misc_feature 20..21: note = DNA
cagacatgctcactgagattt

SEQ ID NO: 15 - DNA, 34 bp, synthetic construct
  source 1..34: mol_type = other DNA
agatcggaagagcacacgtctgaactccagtcac

SEQ ID NO: 16 - DNA, 33 bp, synthetic construct
  source 1..33: mol_type = other DNA
agatcggaagagcgtcgtgtagggaaagagtgt

SEQ ID NO: 17 - RNA, 48 nt, synthetic construct
  source 1..48: mol_type = other RNA
aaaaaaaaaaaaaaaaaaaaaaaaaaaaaaaaaaaaaaaaaaaaaaaa

SEQ ID NO: 18 - DNA, 48 bp, synthetic construct
  source 1..48: mol_type = other DNA
tttttttttttttttttttttttttttttttttttttttttttttttt

(19) United States
(12) Patent Application Publication
(10) Pub. No.: US 2024/0417736 A1
(43) Pub. Date: Dec. 19, 2024

(54) INHIBITION OF GLUTARYL-COA DEHYDROGENASE FOR THE TREATMENT OF MELANOMA

(71) Applicant: Sanford Burnham Prebys Medical Discovery Institute, La Jolla, CA (US)

(72) Inventors: Ze'ev A. RONAI, Fallbrook, CA (US); Sachin VERMA, San Diego, CA (US)

(21) Appl. No.: 18/764,707

(22) Filed: Jul. 5, 2024

Related U.S. Application Data
(63) Continuation of application No. PCT/US2023/010318, filed on Jan. 6, 2023.
(60) Provisional application No. 63/297,388, filed on Jan. 7, 2022.

(51) Int. Cl. C12N 15/113; A61P 35/00

(52) U.S. Cl. CPC C12N 15/1137; A61P 35/00; C12N 2310/141; C12N 2310/531; C12Y 103/08006

(57) ABSTRACT

Addiction to particular metabolic pathways is common to tumor cells. Disclosed herein are compounds and methods to modulate metabolic signaling facilitated by a glutaryl-CoA dehydrogenase (GCDH) protein for the treatment of cancer. In some embodiments, the compounds can reduce GCDH protein expression or activity and reduce the progression of cancers expressing GCDH.

### CROSS-REFERENCE TO RELATED APPLICATION

This application is a continuation of International Patent Application No. PCT/US2023/010318, filed Jan. 6, 2023, which claims the benefit of U.S. Provisional Application No. 63/297,388, filed on Jan. 7, 2022, each of which is hereby incorporated by reference in its entirety.

### STATEMENT AS TO FEDERALLY SPONSORED RESEARCH

This invention was made with government support under R35 CA197465 awarded by the National Institutes of Health. The government has certain rights in the invention.

### CROSS-REFERENCE TO RELATED APPLICATION

The instant application contains a Sequence Listing which has been submitted electronically in XML format and is hereby incorporated by reference in its entirety. Said XML copy, created on Mar. 13, 2023, is named 42256-795_601_SL.xml and is 18,468 bytes in size.

### BACKGROUND

Metabolic pathways that supply energy to normal cells are often rewired in transformed cells to secure sufficient energy for rapid tumor cell proliferation. Among cancer relevant metabolic pathways are those functioning in uptake and utilization of amino acids. Addiction to particular metabolic pathways is common to tumor cells. Tumor cell addiction can be reversed by targeting a specific pathway or restricting availability of a particular amino acid.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, described herein is a method of treating a condition, the method comprising administering to a subject in need thereof a therapeutically-effective amount of a compound, wherein the compound modulates a glutaryl-CoA dehydrogenase (GCDH) protein activity.

### BRIEF DESCRIPTION OF THE DRAWINGS

FIGS. 1A-1J show that GCDH is required for melanoma cell survival.

FIGS. 2A-2F show that GCDH inhibition promotes UPR-dependent cell death signaling.

FIGS. 3A-3E show that GCDH loss in melanoma cells increases NRF2 levels and enhances UPR/cell death signaling.

FIGS. 4A-4E show that lysine glutarylation increases NRF2 stability by attenuating KEAP1 binding.

FIGS. 5A-5B show that GCDH inhibition using inducible shRNA attenuates melanoma proliferation and tumorigenesis.

FIGS. 6A-6F show that GCDH inhibition promotes apoptosis in melanoma cells.

FIGS. 7A-7H show that GCDH expression coincides with patient outcome in melanoma.

FIGS. 8A-8F show that DHTKD1 inhibition rescues gene expression changes and cell death phenotypes seen following GCDH inhibition.

FIGS. 9A-9E show that GCDH activity in melanoma cells antagonizes NRF2-mediated activation of ATF3/4 downstream apoptotic signaling.

FIGS. 10A-10G show that lysine glutarylation increases NRF2 stability and antagonizes KEAP1 binding.

### DETAILED DESCRIPTION OF THE INVENTION

Metabolic pathways that supply energy to normal cells are often rewired in transformed cells to secure sufficient energy for rapid tumor cell proliferation. Among cancer relevant metabolic pathways are those functioning in uptake and utilization of amino acids, including glucose and glutamine, among other amino acids hubs. As a critical source of cellular wealth, amino acid catabolism is implicated in key homeostatic activities, including control of redox levels, ATP production, nucleotide biosynthesis and lipogenesis. Common to these is tight control of fine-tuned signal transduction pathways, which are governed by spatial and temporal post translational modifications by various metabolites derived from amino acid catabolism (i.e., methylation, acetylation, malonylation, succinylation, and glutarylation).

Addiction to particular metabolic pathways is common to tumor cells and often serves as their Achilles Heel in terms of vulnerability. Tumor cell addiction can be reversed by targeting a specific pathway or restricting availability of a particular amino acid. Examples include strategies to limit glutamine, asparagine, serine/glycine or methionine; however, these approaches have met limited success, as sensing mechanisms promote compensatory growth factor or autophagic signaling.

Although tumors have been the focus of these analyses, metabolic cues are also critical in the tumor microenvironment, including for anti-tumor immunity. Limiting availability of a particular amino acid to tumor cells can curtail an anti-tumor immune response, for example, in glucose, glutamine and asparagine metabolism. Combination therapies, in which metabolic signaling is blocked in the presence of other drugs that target oncogenic signaling can be used to address limited availability of amino acids. Limiting asparagine uptake while inhibiting MAPK signaling can efficiently inhibit growth of pancreatic and melanoma tumor cells, with limited impact on immune cell function. Targeting protein methyl transferase 5 (PRMT5) coupled with PD1 therapy can be advantageous in targeting cold melanoma tumors. It is critical to characterize metabolic pathways that underlie cancer cells' ability to adapt to environmental or therapeutic pressure and also to identify their synthetic lethal partners.

The essential amino acids lysine and tryptophan serve as building blocks for proteins and function in acetyl-CoA production and immunosuppression, activities critical for cancer cell survival. Lysine and tryptophan are degraded via a common pathway in which the dehydrogenase DHTKD1 catalyzes synthesis of the intermediate glutaryl CoA. Glutaryl-CoA Dehydrogenase (GCDH) then converts glutaryl CoA to crotonyl CoA, which is metabolized to acetyl CoA to enter the TCA cycle. GCDH functions in TCA cycle-independent pathways and restricts lysine glutarylation by promoting glutaryl CoA breakdown. The lysine catabolism pathway is dispensable for normal development and tissue homeostasis, but GCDH is necessary for survival during protein catabolism. Coincident with GCDH loss and elevated lysine glutarylation is stabilization of the transcription factor NRF2, a master regulator of the cellular stress response implicated in cellular oxidative, nutrient, UPR/ER and metabolic stress responses. Enrichment of ER stress and unfolded protein response (UPR) genes was also observed as part of Keap1-mutant-specific vulnerabilities in lung adenocarcinoma tumor harboring hyperactive NRF2. NRF2 functions as both an oncogene and a tumor suppressor. Disclosed herein is a method of treating a condition, the method comprising administering to a subject in need thereof a therapeutically-effective amount of a compound that modulates glutaryl-CoA dehydrogenase (GCDH) protein activity. In some embodiments, the compound comprises a nucleic acid. In some embodiments, the compound comprises a ribonucleic acid. In some embodiments, the ribonucleic acid comprises a sequence that is at least 80% identical to SEQ ID NO: 13 (CCAAGACCUGGAUCACGAA[dT][dT]) or SEQ ID NO: 14 (CAGACAUGCUCACUGAGAU[dT][dT]).

### Pharmaceutical Compositions of the Invention.

A pharmaceutical composition of the invention can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds of the invention can be applied topically to the skin, or a body cavity, for example, oral, vaginal, bladder, cranial, spinal, thoracic, or pelvic cavity of a subject. The compounds of the invention can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 4 mg/kg, about 100 mg/kg to about 300 mg/kg, or about 150 mg/kg to about 200 mg/kg.

### Methods of Use

In some embodiments, described herein is a method of treating a condition, the method comprising administering to a subject in need thereof a therapeutically-effective amount of a compound, wherein the compound modulates a glutaryl-CoA dehydrogenase (GCDH) protein activity. In some embodiments, the administering decreases GCDH protein activity in the subject. In some embodiments, the administering decreases a GCDH protein expression level in the subject. In some embodiments, the administering decreases GCDH protein enzyme activity in the subject. In some embodiments, the compound comprises a nucleic acid. In some embodiments, the compound comprises a ribonucleic acid. In some embodiments, the ribonucleic acid comprises a microRNA. In some embodiments, the ribonucleic acid comprises a short hairpin RNA. In some embodiments, the ribonucleic acid comprises a small interfering RNA. In some embodiments, the ribonucleic acid comprises a sequence that is at least about 80% identical to SEQ ID NO: 13. In some embodiments, the ribonucleic acid comprises a sequence that is at least about 80% identical to (SEQ ID NO: 14. In some embodiments, the ribonucleic acid comprises a sequence that is at least about 90% identical to SEQ ID NO: 13. In some embodiments, the ribonucleic acid comprises a sequence that is at least about 90% identical to SEQ ID NO: 14. In some embodiments, the ribonucleic acid comprises a sequence that is about 100% identical to SEQ ID NO: 13. In some embodiments, the ribonucleic acid comprises a sequence that is about 100% identical to SEQ ID NO: 14.

In some embodiments, compounds of the invention can be used to treat cancer in a subject. A compound of the invention can, for example, slow the proliferation of cancer cell lines, or kill cancer cells. Non-limiting examples of cancer that can be treated by a compound of the invention include: acute lymphoblastic leukemia, acute myeloid leukemia, adrenocortical carcinoma. AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

In some embodiments, described herein is a method of treating a cancer. In some embodiments, the cancer is a glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, skin cancer, or blood cancer. In some embodiments, the cancer is a skin cancer. In some embodiments, the cancer is a basal cell carcinoma. In some embodiments, the cancer is a squamous cell carcinoma. In some embodiments, the cancer is a melanoma.

In some embodiments, the compounds of the invention show non-lethal toxicity.

### EXAMPLES

### Example 1: Methods and Materials

Animal studies: Animal experiments were performed in compliance with the IACUC guidelines. The study was performed with all relevant ethical regulations regarding animal research. The xenograft model was established using A375 cells expressing either control or shRNA targeting GCDH using doxycycline inducible PLKO-1 vector. NOD/SCID (NOD.CB17-Prkdcscid/J) mice were used. Eight-week-old male C57BL/6 mice were injected subcutaneously in the flank with 1×10⁶ A375 cells. For in vivo GCDH KD experiments, mice were fed rodent chow containing 200 mg/kg doxycycline (Dox diet) to induce GCDH-KD. Mice were sacrificed upon signs of morbidity resulting from tumor growth. Tumor volume was measured with linear calipers and calculated using the formula: (length in mm×width in mm)×½. After the mice were sacrificed, tumors were frozen or fixed in Z-Fix. Snap-frozen tumors were utilized for protein extraction for further analysis.

Cell culture and reagents: Cancer cell lines (breast: SKBR3 and MCF7; prostate: PC-3 and DU145; liver: SK-HEP1 and PLC and melanoma: A375 and 1205LU) were obtained from ATCC. WM1346 and WM1366 and WM3629 melanoma cell lines, and the UACC-903 melanoma cell line were gifted. All cell lines were cultured in Dulbecco's modified Eagle's medium (DMEM), and supplemented with 5% fetal bovine serum and penicillin-streptomycin. All cells were grown at 37° C. in a humidified atmosphere containing 5% carbon dioxide. Amino acids (L-lysine, L-arginine), proteasomal inhibitor (MG132), ATM Inhibitor (KU-55933) and p53 inhibitor (Pifithrin-α) were purchased. 1×10⁵ cells were seeded overnight (0/N) per well in 6-well plates. Negative control (NT-siRNA) or si-RNA targeting the transcript of interest was transfected utilizing jetPRIME® transfection reagent. Following siRNAs were used: si-GCDH (SEQ ID NO: 13 and SEQ ID NO: 14), siDHTKD1 (SASI_Hs02_00352234 and SASI_Hs02_00352235), si-ATF4 (SASI_Hs02_00332313), si-ATF3 (NM_001030287), si-DDIT3 (SASI_Hs01_00153013), si-CHAC1 (SASI_Hs01_00146246), si-NRF2 (SASI_Hs01_00182393), si-AASS (SASI_Hs02_00340239 and SASI_Hs01_00016127) si-ADAT (SASI_Hs01_00091485 and SASI_Hs01_00091486) siECHS1 (SASI_Hs01_00085563 and SASI_Hs02_00336896) and the negative control si-RNA (NT-siRNA; SIC001). Plasmid encoding HA-NRF2 (pLV-mCherry-CMV HA-NRF2) was synthesized by Vector builder.

Cell proliferation and viability: For cell proliferation, 0.3-0.5×10⁵ cells were seeded O/N in triplicate in 6-well plate. Following treatment for the specified duration, cells were trypsinized and the cell count was determined with Neubauer hemocytometer. To measure cell viability, cells were washed twice with cold PBS and fix for 10 minutes with ice-cold 100% methanol. Cells were then incubated with 0.5% crystal violet solution in 25% methanol for 10 minutes at room temperature. Crystal violet solution was removed, and cells washed in water several times until the dye was removed. The culture plates were then dried at room temperature. For quantitation, 2 mL 10% acetic acid to each well (6 well) was incubated for 20 min with shaking (extraction). 0.5-1 mL of extracted stain was diluted 1:4 in water (dilution flexible based on signal intensity) followed by quantification absorbance at 590 nm. The resulting readings were normalized as compared to absorbance from respective control cells.

SubG1 DNA content analysis to quantify apoptotic population: SubG1 DNA content analysis was performed for determination of apoptotic population, analyzed by propidium iodide staining. Briefly, 1×10⁶ cells were washed twice with cold PBS and fixed in 70% ethanol in PBS at 4° C. overnight. Cells were washed, pelleted by centrifugation, and treated with RNase A (100 μg/mL) and propidium iodide (40 μg/mL) at room temperature for 30 min. Cell cycle distribution was assessed by flow cytometry, and data was analyzed using FlowJo software.

Immunoblotting: Total protein was extracted in Laemmli buffer, fractionated by SDS polyacrylamide gels and transferred to PVDF membranes. After blocking with 5% non-fat dry milk, the membranes were incubated with primary antibodies overnight at 4° C. Afterwards, 2 hr incubation with HRP-conjugated secondary antibodies was performed. Following chemiluminescence reaction, the protein signal was visualized using the ChemiDoc imaging system.

qPCR analysis: 1×10⁵ cells were seeded O/N per well in 6-well plates. Following treatment for 72 hr, total RNA was extracted using RNeasy Mini Kit. cDNA was synthesized using oligo(dT) and random primers, and qPCR analysis was performed with SYBR Green. Primers were designed using the PrimerQuest tool and Primer Bank. Actin was used as an internal control. Primer efficiency was measured in preliminary experiments, and amplification specificity was confirmed by dissociation curve analysis. TABLE 1 shows the sequence of the primers used.

TABLE 1

| SEQ ID | | |
|---|---|---|
| NO: | Primer | Sequence |
| 1 | ATF3-F | CCTCTGCGCTGGAATCAGTC |
| 2 | ATF-R | TTCTTTCTCGTCGCCTCTTTTT |
| 3 | ATF4-F | GTATGAGCCCAGAGTCCTATCT |
| 4 | ATF4-R | CACATTGACGCTCCTGACTATC |
| 5 | CHACI-F | GAACCCTGGTTACCTGGGC |
| 6 | CHACI-R | CGCAGCAAGTATTCAAGGTTGT |
| 7 | DDIT3-F | GGAAACAGAGTGGTCATTCCC |
| 8 | DDIT3-R | CTGCTTGAGCCGTTCATTCTC |
| 9 | GCDH-F | CGTCCCGAGTTTGACTGGC |
| 10 | GCDH-R | GATGCGAGGCATGAGTCTCT |
| 11 | β-Actin-F | CATGTACGTTGCTATCCAGGC |
| 12 | β-Actin-R | CTCCTTAATGTCACGCACGAT |

Intracellular glutarate quantification: Cell extraction and GC-MS analysis for Glutarate quantification were performed. Intracellular metabolite amounts are expressed in nmol per cell sample (cells from one well of six-well plates; approximately 0.5×10⁶-1.0×10⁶ cells).

Antibodies: The following antibodies were used: NRF2 (D1Z9C) (dilution, 1:1,000), ATF4 (D4B8) (dilution, 1:1.000), CHOP (L63F7) (dilution 1:1,000), p21 Waf1/Cip1 (12D1) (dilution 1:1,000), HO1 (E9H3A) (dilution, 1:1,000), Cleaved Caspase-3 (Asp175) (5A1E) (dilution 1:1,000) and HRP-conjugated anti-Mouse (dilution 1:10.000) and anti-Rabbit (dilution, 1:10,000) antibodies. Bcl-2 (C-2) (dilution 1:1,000) Mcl-1 Antibody (22) (dilution, 1:1,000) and HSP90 (F-8) (dilution, 1:5,000); GCDH/GCD antibody (ab232774) (dilution 1:10,000) DHTKD1 antibody (ab230392) (dilution 1:10,000) antibodies; Pan Anti-glutaryllysine antibody; and CHAC1 (dilution 1:500) antibody.

Cycloheximide chase assay: Briefly, cycloheximide (50 μg/mL) was added to cells for indicated times, and cell lysates were analyzed with indicated antibodies.

Immunoprecipitation: The protein-protein interaction for endogenous protein was studied by using the Co-Immunoprecipitation Kit. For overexpressed HA-tag proteins, HEK293T cells were transfected with plasmids encoding the HA-NRF2 (pLV-mCherry-CMV HA-NRF2). After 48 hours of transfection, cells were treated with MG132 for 4 hours and washed with PBS. Cells were lysed in IP milder Lysis/Wash Buffer (0.025M Tris, 0.15M NaCl, 0.00M EDTA, 1% NP-40, 5% glycerol; pH 7.4) and HA-antibody-conjugated agarose resin was added. The cells were rotated overnight at 4° C. After incubation, the resin was pelleted and washed with IP Lysis/Wash Buffer. The resin was then boiled twice in SDS-PAGE loading buffer for 5 min and analyzed by western blotting. For detection of glutarylated NRF2 from cell fractions (MF-membrane, NF-nuclear and CF-cytoplasmic), first cell fractionation was carried out using a Cell Fractionation Kit. Equal amounts of protein from purified fractions were incubated with HA beads overnight at 40° C.

In vitro glutarylation, KEAP1 binding and ARE-EMSA: HEK293T cells expressing HA-NRF2 were lysed in RIPA buffer (20 mM Tris-HCl (pH 7.5), 150 mM NaCl, 1 mM Na₂ EDTA, 1 mM EGTA, 1% NP-40, 1% sodium deoxycholate, 2.5 mM sodium pyrophosphate, 1 mM glycerophosphate, 1 mM Na3 VO4, and 1 μg/mL leupeptin) containing N-Ethylmaleimide (NEM). The lysates were incubated with HA binding beads and washed with RIPA lysis buffer to retain HA-NF2 on beads and subjected to in vitro glutarylation reaction. For in vitro Glutarylation of NRF2, purified HA-NRF2 in glutarylation buffer (50 mM HEPES [pH 8.0], 150 mM NaCl. and protease inhibitors) were mixed with different concentration of glutaryl-CoA or glutaric acid to form glutaryl-NRF2 (K Glu-NRF2) and control HA-NRF2, respectively. The reactions were incubated in an Eppendorf Thermomixer for 4 hr at 37° C. at 400 rpm. To minimize condensation, samples were briefly centrifuged every hour during incubation. The washed HA-beads were then used for KEAP1 binding or EMSA directly. For KEAP1 binding assay, beads were incubated with cell extract (for KEAP1 binding) in IP lysis/wash buffer. After subsequent washing steps, the beads were boiled twice in SDS sample buffer. The proteins were separated on SDS/PAGE and immunoblotted with indicated antibodies as described. For ARE-EMSA, equal amounts of HA-NRF2 and K-Glu NRF2 were subjected to binding with ARE-Biotin labelled DNA probe (NRF2(ARE) EMSA Kit).

RNA-Seq data analysis: Illumina Truseq adapter, polyA, and polyT sequences were trimmed with cutadapt v2.3 using parameters “cutadapt-j 4-m 20-interleaved-a AGATCGGAAGAGCACACGTCTGAACTCCAGTCAC-AAGATCGGAAGAGCGTCGTGTAGGGAAAGAGTGT Fastq1 Fastq2| cutadapt-interleaved-j 4-m 20-a″A⁴⁸″-A″A⁴⁸″-|cutadapt-j 4-m 20-a″T⁴⁸″-A″T⁴⁸″-”. Trimmed reads were aligned to human genome version 38 (hg38) using STAR aligner v2.7.0d 0221 55 with parameters according to ENCODE long RNA-seq pipeline. Gene expression levels were quantified using RSEM v1.3.1 56. Ensemble gene annotation version 84 was used in the alignment and quantification steps. RNA-seq sequence, alignment, and quantification quality was assessed using FastQC v0.11.5 and MultiQC v1.8 57. Biological replicate concordance was assessed using principal component analysis (PCA) and pair-wise pearson correlation analysis. Lowly expressed genes were filtered out by applying the following criterion: estimated counts (from RSEM)≥number of samples*5. Filtered estimated read counts from RSEM were compared using the R Bioconductor package DESeq2 v1.22.2 based on generalized linear model and negative binomial distribution. Genes with Benjamin-Hochberg corrected p-value<0.05 and fold change≥2.0 or ≤−2.0 were selected as differentially expressed genes. Differentially expressed genes were analyzed using Ingenuity Pathway Analysis. RNA-seq figures were plotted using ggplot2 (H. Wickham, ggplot2: Elegant Graphics for Data Analysis) and ComplexHeatmap.

TCGA survival analysis: Gene expression (RNA-seq) and clinical data from TCGA Pan-Cancer 2018 were downloaded from cBioPortal. Survival analysis was performed in R version 4.0.2 using survival. A Package for Survival Analysis in R. R package version 3.2-7; survminer (Alboukadel Kassambara, Marcin Kosinski and Przemyslaw Biecek); survminer: Drawing Survival Curves using ‘ggplot2’. R package version 0.4.8.999; maxstat (Torsten Hothom; maxstat: Maximally Selected Rank Statistics. R package version 0.7-25. Optimal cutpoint for the categorization of TCGA samples as ‘high’ and ‘low’ GCDH expressors in each cancer type was determined using surv_cutpoint( ) and surv_categorize( ) functions from survminer package.

Measurement of cellular respiration: siRNA transfected A375 cells were plated at a density of 10,000 per well in a Seahorse XFp culture plate and cultured overnight before changing the medium to Seahorse XF base medium containing 1 g/l glucose and 2 mM glutamine, pH 7.4, and assaying oxygen consumption and extracellular acidification rates in the Seahorse XFp with successive additions of 1.5 mM oligomycin and 1.0 mM FCCP.

Statistical Analysis: Statistical significance between two groups was assessed by the unpaired Student's t-test. Ordinary one-way ANOVA was used to analyze more than two groups. Two-way ANOVA was utilized to analyze cell proliferation at multiple timepoints. GraphPad Prism 5 software Graphpad 8.0.0 was used for to perform all statistical calculations.

### Example 2: GCDH is Required for Melanoma Cell Survival

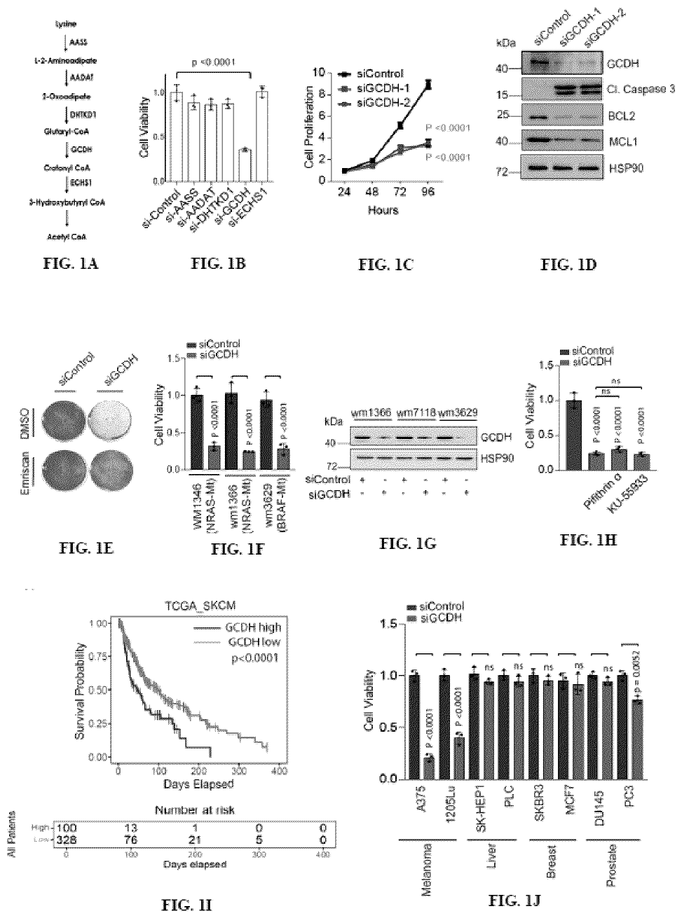

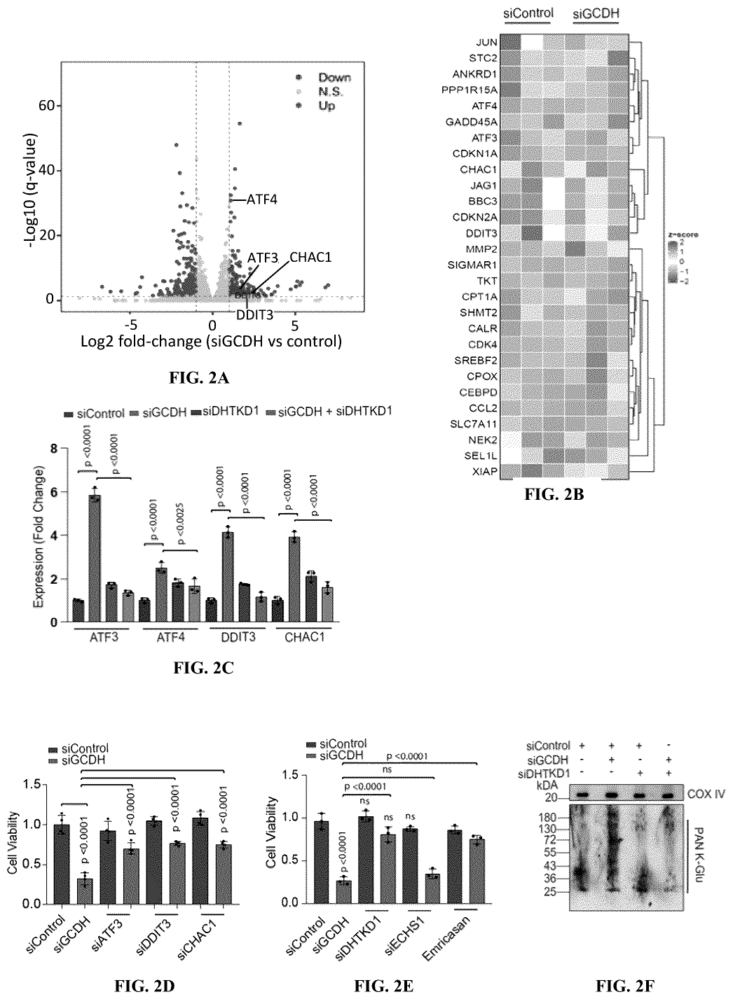

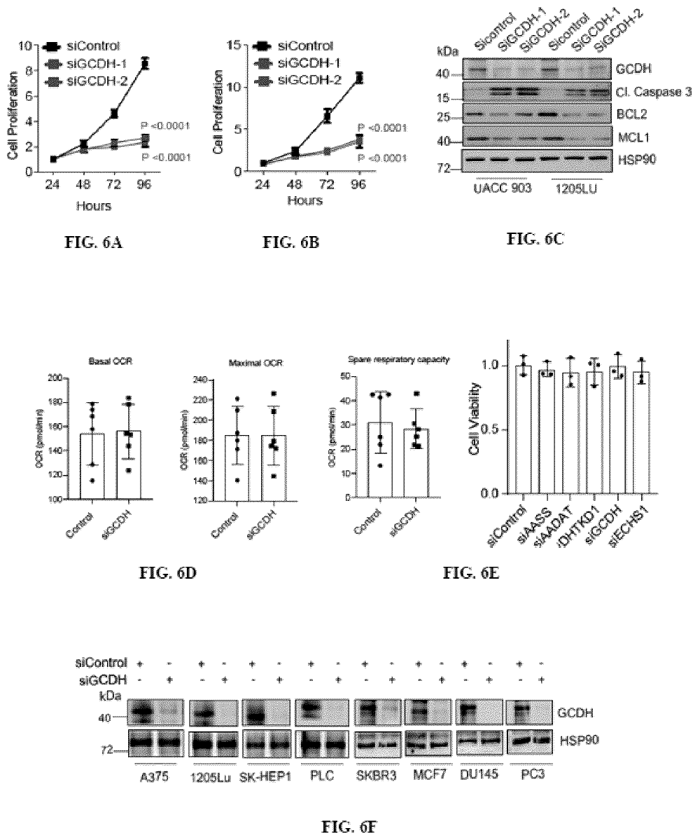

Lysine restriction can completely block cancer cell growth in colon carcinoma cell lines. The acetyl CoA generated from lysine catabolism can drive liver metastasis of colorectal cancer. To directly assess the importance of the lysine catabolism pathway for melanoma cell viability the requirement of each of the component in this metabolic pathway for melanoma cell survival was monitored. siRNA inhibiting the expression of aminoadipic semialdehyde synthase (AASS). Kynurenine/alpha aminoadipate aminotransferase (AADAT), Dehydrogenase E1 and transketolase domain containing 1 (DHTKD1), GCDH or Enoyl-CoA Hydratase, Short Chain 1 (ECHS1), components of the lysine catabolism pathway in A375 melanoma cells were used (FIG. 1A). Only GCDH knock down (KD) resulted in a pronounced cell death in A375 cells (FIG. 1B). The results were confirmed using two independent siRNAs and recapitulated in two additional melanoma cell lines (UACC903, and 1205LU) (FIG. 1C; FIGS. 6A and 6B). Correspondingly, GCDH KD upregulated cleaved caspase 3 and downregulated levels of the antiapoptotic markers BCL2/MCL1 in all three melanoma cell lines (FIG. 1D; FIG. 6C). Apoptotic signaling induced by GCDH KD was effectively reversed by treatment with the caspase inhibitor Emriscan (FIG. 1E; FIG. 2E). Notably, melanoma cell dependency on GCDH was found to be independent of mutational status (FIGS. 1F and G), indicating a metabolic signaling pathway that is uncoupled from BRAF or NRAS signaling pathways. These data demonstrate the requirement of GCDH for melanoma cell survival.

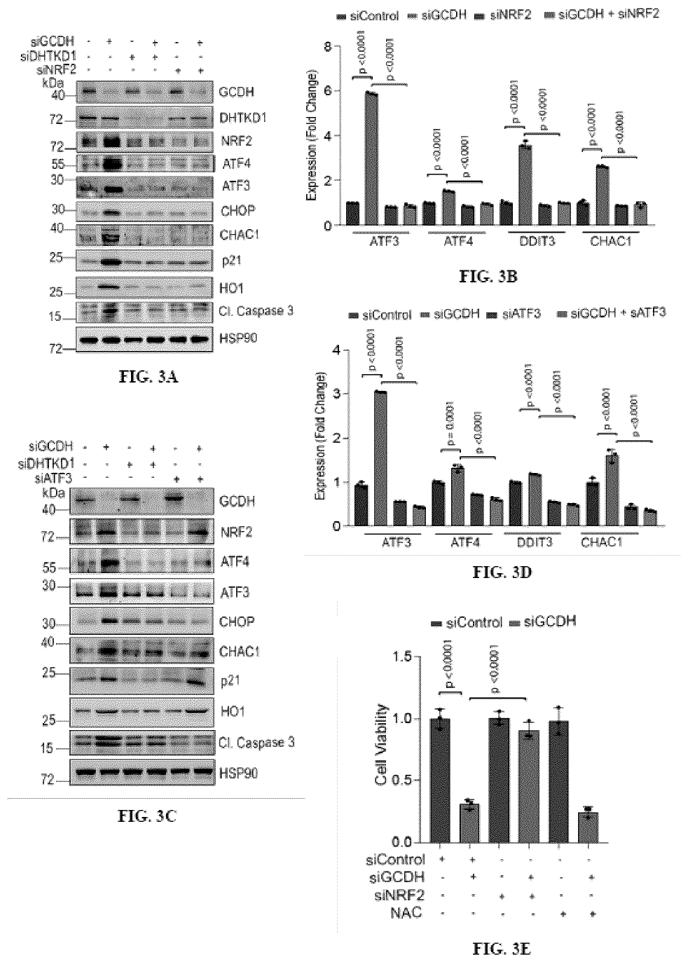

Cell death seen upon GCDH KD in A375 melanoma cells (p53 wt) could not be rescued upon pharmacological inhibition of either p53 (Pifithrin-α) or ATM (KU-55933) nor was it affected upon treatment with the ROS scavenger N-acetylcysteine (FIG. 1H and FIG. 3E). The data showed that melanoma dependency on GCDH was independent of DNA damage or oxidative stress. The ability of GCDH inhibition to alter mitochondrial biogenesis or cellular respiration was studied. A GCDH KD study was performed, and basal, maximum oxygen consumption rate (OCR) and spare respiratory capacity were measured. None of these were altered significantly in GCDH KD versus control cells (FIG. 6D), indicating a lack of effect on mitochondrial biogenesis and respiration. Notably, neither GCDH nor any other component of the lysine catabolism pathway was required for cell viability in the non-transformed and immortalized melanocyte cell line H3A (FIG. 6E). This observations were consistent with GCDH, and DHTKD1-KO mouse phenotype where GCDH activity was found to be dispensable for overall viability and growth of normal cells.

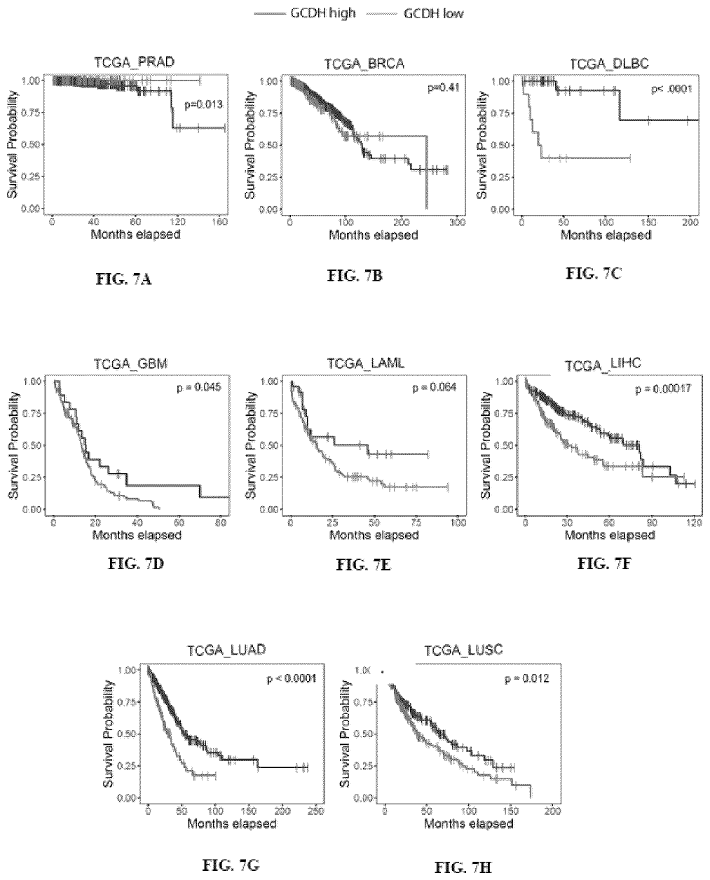

Evaluation of melanoma patient samples revealed that higher GCDH expression was associated with decreased patient survival (FIG. 1I), whereas patients with low levels of GCDH expression exhibited a significant survival advantage, compared with those with high levels. Notably, such correlation was not seen in patients with other tumor types (FIGS. 7A-7H).

Consistent with patient data, inhibiting GCDH expression in melanoma lines, but not in liver, breast or prostate cancer cultures, promoted notable cell death (FIG. 1J and FIG. 6F), highlighting the specificity of GCDH signaling in melanoma. These data highlight the importance of GCDH for melanoma cell survival.

FIGS. 1A-1K show that GCDH is required for melanoma cell survival. FIG. 1A illustrates a schematic representation of enzymes involved in lysine catabolic pathway. FIG. 1B shows siRNA targeting AASS, AADAT, DHTKD1, GCDH, ECHS1 or control sequence were transfected into A375 melanoma cells by Jetprime for 96 hours. Cell viability was then measured by quantifying crystal violet staining. FIG. 1C shows cell growth upon GCDH knock down using two independent siRNAs for 0-96 hr in A375 cell line. Cell growth was analyzed by cell counting at indicated time points. FIG. 1D shows A375 cells were transfected with siRNA against GCDH, and western blot analysis was done using indicated antibodies. FIG. 1E shows cell viability assay of control or GCDH KD A375 cells treated with the caspase inhibitor Emriscan (10 μM for 48 hr). FIG. 1F shows cell viability assay of indicated cells 96 hr after transfection with siRNAs targeting GCDH. Cell viability was measured by quantifying crystal violet staining. FIG. 1G shows western blot analysis confirming GCDH KD as described in D. FIG. 1H shows analysis of cell viability upon GCDH-KD alone or in combination with treatment with Pifithrin-α or KU-55933 in A375 cells. FIG. 1I shows survival analysis of GCDH expression in melanoma patients using TCGA. Number of total patients, n=428, n (GCDH high)=328 and n (GCDH low)=100. FIG. 1J shows cell viability was measured by quantifying crystal violet staining upon GCDH KD in various cancer cell lines as indicated. FIG. 1K shows Western blot analysis on A375 cells transfected with siRNA targeting GCDH for 96 hours using indicated antibodies. Data are represented as mean±SEM of n=3 independent experiments. Statistical significance (indicated p value or ns—not significant w.r.t control) was calculated using unpaired t-test.

FIGS. 6A-6F show that GCDH inhibition promotes apoptosis in melanoma cells. FIGS. 6A-6B show cell growth upon GCDH knock down using two independent siRNAs for 0-96 hr in UACC903 (FIG. 6A) or 1205LU (FIG. 6B). Cell growth was analyzed by cell counting at indicated time points. FIG. 6C shows UACC903 or 1205LU cells were transfected with siRNA against GCDH for 72 hours, and western blot analysis was done using indicated antibodies. FIG. 6D shows measurement of basal, maximum oxygen consumption rate (OCR) and spare respiratory capacity. siRNA targeting GCDH was transfected into A375 cells before analysis. FIG. 6E shows cell viability assay of immortalized H3A cells, 96 hr after transfection with indicated constructs. Cell viability was measured by quantifying crystal violet staining. Data are presented as the mean±SEM. Statistical significance (indicated p value or ns—not significant w.r.t control) was calculated using unpaired t-test.

FIGS. 7A-7H show that GCDH expression coincides with patient outcome in melanoma. Survival correlation analysis of GCDH expression in various cancer subtypes (prostate adenocarcinoma (PRAD), breast cancer (BRCA), diffuse large B-cell lymphoma (DLBC), glioblastoma (GBM), acute myeloid leukemia (LAML), liver hepatocellular carcinoma (LIHC), lung adenocarcinoma (LUAD), lung squamous cell carcinoma (LUSC) using TCGA.

### Example 3: GCDH Inhibition Promotes UPR-Dependent Cell Death Signaling

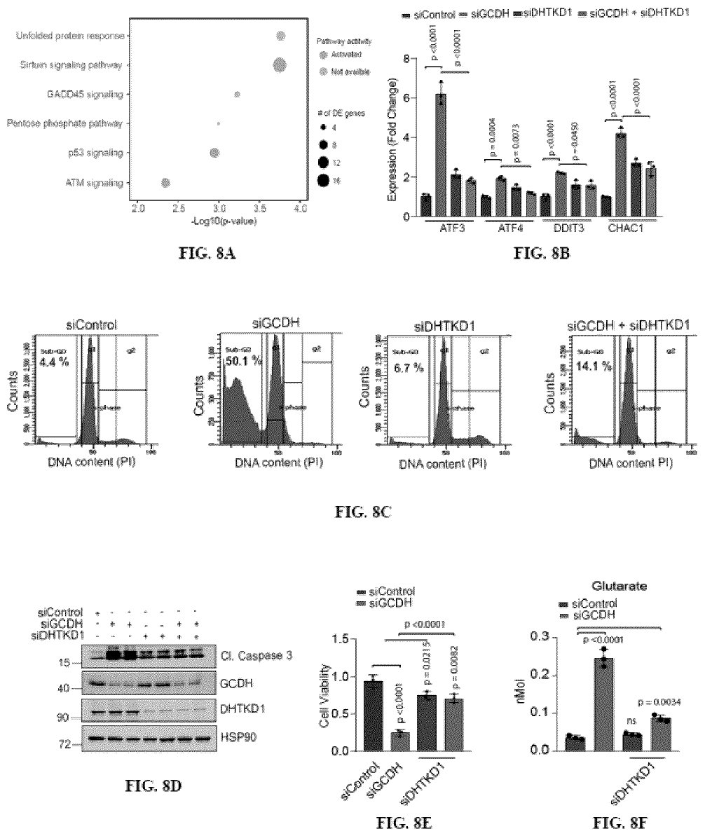

To identify possible mechanisms that underlie cell death induced upon GCDH loss, changes in gene expression were monitored following GCDH KD in melanoma cells. RNAseq analysis followed by IPA assessment of signaling pathways that were differentially expressed upon GCDH KD identified UPR. sirtuin, GADD45, pentose phosphate, p53 and ATM signaling pathways (FIG. 8A). Among differentially expressed genes were upregulation of genes controlled by ATF3 and ATF4 signaling implicated in UPR-induced cell death (i.e., DDIT3, —CHAC1, and GADD45a), and genes implicated in cell cycle inhibition and tumor suppression (i.e., CDKN1A and CDKN2B45-47 (FIGS. 2A and 2B). qPCR analysis of gene signatures performed in both A375 and UACC903 cells confirmed upregulation of apoptotic UPR signaling as reflected by increased levels of ATF3, ATF4, CHOP and CHAC1 transcripts upon GCDH KD (FIGS. 2C and 8B) in A375 and UACC903 cells respectively. Inhibition of either ATF3. DDIT3 or CHAC1 in A375 melanoma cells subjected to GCDH KD, effectively attenuated the degree of cell death induced upon GCDH KD (FIG. 2D). The data demonstrate that GCDH loss in melanoma cells induces UPR-dependent cell death pathways regulated by the ATF3-DDIT3-CHAC1 apoptotic cascade44.

Consistent with apoptotic signaling observed at molecular level (FIGS. 1D and 6C). GCDH KD in A375 cells led to a 50.1% increase in cells exhibiting DNA fragmentation, indicative of cell death, relative to controls (4.4%) (FIG. 8C). Significantly, the degree of cell death seen upon GCDH KD was decreased to 14.1% by concomitant KD of DHTKD1, which is upstream of GCDH in the pathway (FIGS. 1A, 8C and 8D). While DHTKD1 KD also effectively rescued viability of BRAF mutant 1205LU melanoma cells subjected to GCDH KD, (FIG. 8E), KD of ECHS1, which is downstream of GCDH (FIG. 1A), did not alter the degree of cell death seen in the presence of GCDH KD (FIG. 2E). Given that GCDH inhibition promotes glutaryl-CoA accumulation and increased protein glutarylation in mitochondria, protein glutarylation in GCDH KD cells was assessed. Relative to control siRNAs, GCDH KD promoted increased glutarylation of proteins in mitochondrial extracts (FIG. 2F). Accordingly, levels of glutarate, a metabolite produced by glutaryl-CoA, increased in GCDH KD A375 cells (FIG. 8F). Changes seen in both glutarylation and glutarate levels were largely rescued upon DHTKD1 KD (FIGS. 2F and 8F). Levels of ATF3, ATF4, DDIT3 and CHAC1 transcripts were also attenuated upon combined KD of DHTKD1 and GCDH (FIG. 2C), rescuing changes seen upon GCDH KD alone. These observations suggest that GCDH controls levels of protein glutarylation, which in turn regulate UPR cell death signaling.

FIGS. 2A-2F show that GCDH inhibition promotes UPR-dependent cell death signaling. FIG. 2A shows volcano plot showing elevated expression of molecules controlling UPR mediated cell death cascade (ATF3, ATF4, DDIT3 and CHAC1), identified by RNA-seq analysis. FIG. 2B shows Heatmap representing differential expression of ATF3/4 downstream targets in GCDH-KD A375 cells identified by RNA-seq analysis. FIG. 2C shows RT_qPCR validation of ATF3, ATF4, DDIT3 and CHAC1 in A375 cells transfected with indicated siRNAs. FIGS. 2D and 2E show cell viability of A375 cells transfected with indicated siRNAs for 96 hours. Cell viability was analyzed by crystal violet staining and quantitation. FIG. 2F Western blot analysis on mitochondrial extracts from A375 cells using PAN K-Glu-detecting antibody to detect lysine glutarylation 72 hr following transfection with indicated siRNAs. Data are resented as the mean±SEM. Statistical significance (indicated p value or ns—not significant w.r.t control) was calculated using unpaired t-test and two-way ANOVA for FIGS. 2C and 2E.

FIGS. 8A-8F show that DHTKD1 inhibition rescues gene expression changes and cell death phenotypes seen following GCDH inhibition. FIG. 8A shows Gene set enrichment analysis to identify signaling pathway affected upon GCDH KD in A375 cells identified by RNA-seq analysis. FIG. 83 shows RT-qPCR analysis in UACC903 cells for relative expression of ATF3. ATF4, DDIT3 and CHAC1 following GCDH-KD, DHTKD1-KD alone or GCDH-DHTKD1 double KD. FIG. 8C shows SubG0 DNA content analysis by flow cytometry to measure apoptosis in A375 cells. A375 cells were transfected with indicated siRNAs for 72 hours and then harvested for fixation in ethanol and staining with propidium iodide (PI). FIG. 8D shows Western blot analysis to measure GCDH, DHTKD1 and Cl. caspase 3 protein levels in A375 lines following GCDH-KD, DHTKD1-KD alone or GCDH-DHTKD1 double KD. FIG. 8E shows Rescue of cell death in GCDH-KD 1205LU upon DHTKD1-KD. Cell viability was measured by quantifying crystal violet staining. FIG. 8F shows GC-MS analysis to measure glutarate concentrations in A375 cells. Data are presented as the mean±SEM. Statistical significance (indicated p value or ns—not significant w.r.t control) was calculated using unpaired 1-test except for FIGS. 8E-8F and two-way ANOVA for FIGS. 8B and 8E.

### Example 4: GCDH Loss in Melanoma Cells Increases NRF2 Levels and Induces NRF2 Dependent UPR Cell Death

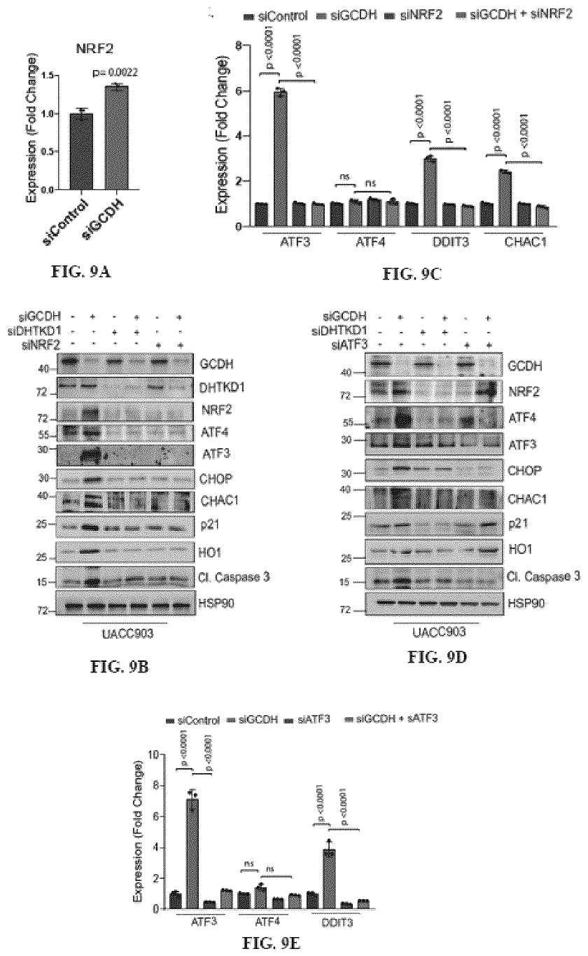

Next, we asked whether NRF2 function is required to induce apoptotic UPR signaling after GCDH KD, given that NRF2 is upregulated in the striatum of GCDH KO mice with high lysine diet and Quinolinic acid induced toxicity 34 and implicated in the regulation of ATF3 and ATF4 transcription. Since NRF2 is regulated at the transcriptional level and protein stability, we monitored potential changes in NRF2 protein and transcript levels after GCDH KD in A375 and UACC903 lines (FIGS. 3A, 9A and 9B). We found that NRF2 mRNA level were only marginally affected upon GCDH KD (FIG. 9A). Whereas elevated NRF2 protein levels coincided with increased abundance of the UPR proteins ATF3, ATF4, DDIT3, CHAC1, caspase 3 (cleaved) and the downstream NRF2 targets HO1 and p21 (FIGS. 3A and S4B). Concomitant KD of NRF2 or DHTKD1 in GCDH KD A375 and UACC903 lines effectively reversed apoptotic UPR signaling seen upon GCDH KD alone, both at the protein (FIGS. 3A and 9B) and transcript (FIG. 3B and FIG. 9C) levels. Given that ATF3 controls DDIT3-CHAC1 signaling, we asked whether ATF3 may mediate phenotypes seen after GCDH loss. ATF3 KD combined with GCDH KD in these lines effectively attenuated cell death seen in the presence of GCDH KD alone (FIG. 2D), similar to the effects seen following DHTKD1 KD (FIG. 2E). ATF3 KD in GCDH KD cells was also accompanied by reduced levels of ATF4, DDIT3, CHAC1 and cleaved caspase 3 protein (FIGS. 3C and 9D). Consistently, decreased levels of ATF4, DDIT3 and CHAC1 transcripts, was observed in cells subjected to ATF3 KD, compared with GCDH KD (FIGS. 3D and 9E). The changes seen upon ATF3 KD phenocopied those observed following DHTKD1 KD, or NRF2 KD in cells that were subjected to GCDH KD, culminating in attenuated apoptotic UPR signaling (FIGS. 2D and 3E). As expected, ATF3 KD alone in melanoma cells had no effect on NRF2 stability or p21 or HO1 expression, suggesting that ATF3 is the primary driver of apoptosis downstream of NRF2 after GCDH inhibition (FIGS. 3C and 9D). Notably, NRF2 KD in the presence of GCDH KD decreased the extent of melanoma cell death seen after GCDH KD alone (FIG. 3E). Collectively, these observations suggest that GCDH controls apoptotic UPR signaling in melanoma cells via elevated abundance of NRF2 protein levels.

FIGS. 3A-3E show that GCDH loss in melanoma cells increases NRF2 levels and enhances UPR/cell death signaling. FIG. 3A western blot analysis of indicated proteins in A375 cells 72 hr following transfection with indicated siRNAs. FIG. 3B shows RT-qPCR analysis of ATF3, ATF4, DDIT3, and CHAC1 expression levels in A375 cells following transfection with indicated siRNAs. FIG. 3C shows western blot analysis of indicated proteins in A375 cells 72 hr following transfection with indicated siRNAs. FIG. 3D shows RT-qPCR analysis of ATF3, ATF4, DDIT3, and CHAC1 expression levels in A375 following transfection with indicated siRNAs. FIG. 3E shows viability assay on A375 cells upon transfected with indicated siRNAs and treated or untreated with NAC (10 mM) for 48 hr. Data are presented as the mean±SEM. Statistical significance (indicated p value or ns—not significant w.r.t control) was calculated using two-way ANOVA for FIGS. 3B, 3D and 3E.

FIGS. 9A-9E show that GCDH activity in melanoma cells antagonizes NRF2-mediated activation of ATF3/4 downstream apoptotic signaling. FIG. 9A shows RT-qPCR analysis of NRF2 mRNA expression upon GCDH KD in A375 cells. FIG. 9B shows Western blot analysis of indicated proteins in UACC903 cells, 72 hr following transfection with various siRNAs. FIG. 9C shows RT-qPCR analysis of ATF3, ATF4, DDIT3, and CHAC1 expression levels in UACC903 cells 72 hr following transfection with indicated siRNAs. FIG. 9D shows Western blot analysis of indicated proteins following in UACC903 cells 72 hr following transfection with siRNAs. FIG. 9E shows RT-qPCR analysis of ATF3, ATF4, DDIT3, and CHAC1 expression levels in UACC903 following transfection with indicated siRNAs. Data are presented as the mean±SEM. Statistical significance (indicated p value or ns—not significant w.r.t control) was calculated using unpaired f-test except for FIG. 9A, and two-way ANOVA for FIGS. 9C and 9E.

### Example 5: GCDH Controls NRF2 Stability

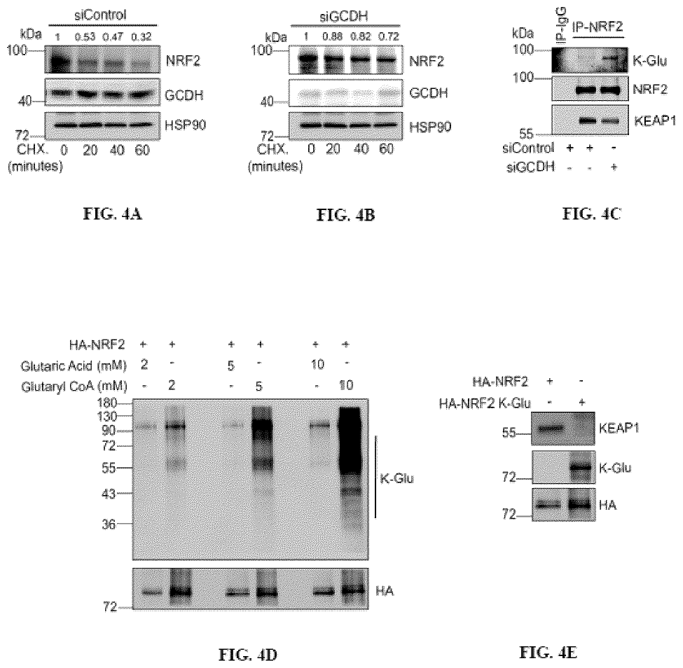

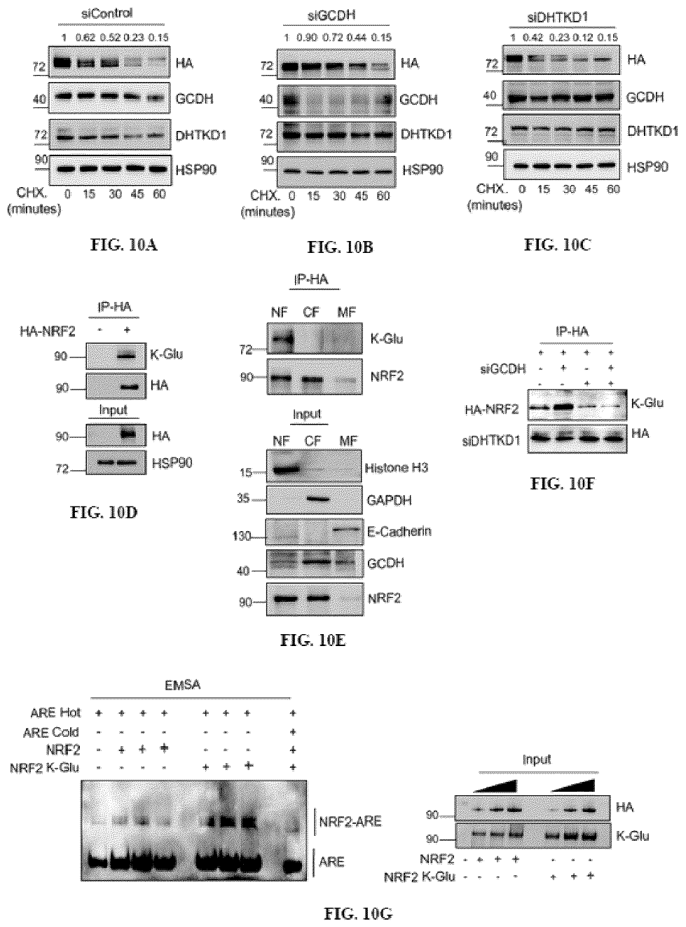

Potential changes in NRF2 stability following GCDH KD in A375 cells were monitored. NRF2 half-life increased following GCDH KD in A375 melanoma cells relative to controls (FIGS. 4A and 4B) as well as in GCDH KD HEK293T cells exogenously expressing HA-NRF2 (FIGS. 10A and 10B). Conversely, DHTKD1 KD decreased NRF2 stability (FIGS. 10A and 10C). As NRF2 stability is tightly controlled by interaction with the ubiquitin ligase KEAP1, possible changes in NRF2/KEAP1 interaction following GCDH KD were assessed. Immunoprecipitation (IP) of endogenous NRF2 from GCDH KD cells revealed lower levels of NRF2-bound KEAP1 relative to control cells (FIG. 4C). Given notable increases in protein glutarylation seen upon GCDH inhibition, the ability of NRF2 glutarylation to alter its interaction with KEAP1 or degree of ubiquitination was studied, given that lysine is the primary glutarylated residue. NRF2 immunoprecipitated from melanoma cells were subjected to immunoblotting with antibodies against lysine glutarylation (K-Glu). While basal levels of NRF2 glutarylation were detected in control A375 cells, those levels notably increased following GCDH KD (FIG. 4C). Likewise, IP of ectopically expressed HA-NRF2 in HEK293T cells followed by immunoblotting with K-Glu antibodies revealed elevated NRF2 glutarylation, compared to controls (FIG. 10D). To confirm NRF2 glutarylation, an in vitro glutarylation assay was performed with purified HA-NRF2 and observed its glutarylation (K-Glu NRF2; FIG. 4D) when incubated with glutaryl-CoA harboring a reactive CoA moiety but not with glutaric acid, which served as control, suggesting that NRF2 undergoes glutarylation in the presence of elevated glutaryl-CoA levels promoted by GCDH KD. The relative amounts of KEAP1-bound to glutarylated NRF2 were determined. IP of HA-NRF2 or K-Glu HA-NRF2 (used as bait) to retain KEAP1 from extracts of A375 cells showed lower interaction of glutarylated NRF2 with KEAP1 (FIG. 4E). Moreover, cell fractionation revealed that glutarylated NRF2 was primarily nuclear (FIG. S5E), implying effects on transcriptional activity. An electrophoretic mobility shift assay was performed to monitor changes in NRF2 binding to the antioxidant response element (ARE), a promoter element in genes which is bound and regulated by NRF2. Relative to the HA-NRF2 control, we observed a notable increase in binding of in vitro glutarylated K Glu HA-NRF2 to the ARE (FIG. 10G), suggesting increased NRF2 affinity for the promoter sequence. RNAseq data performed in melanoma cells after GCDH inhibition confirmed a gene expression signature (FIGS. 2A and 8A) different from that seen in control cells but consistent with an NRF2-activated UPR signature. These data suggest that GCDH control of NRF2 glutarylation not only determines its stability but also modulates its binding to DNA.

FIGS. 4A-4E show that lysine glutarylation increases NRF2 stability by attenuating KEAP1 binding. FIGS. 4A-4B show cycloheximide (CHX) chase to check half-life of endogenous NRF2 in A375 cells transfected with siRNA targeting GCDH. Western blot was performed on lysate from A375 transfected with siControl (FIG. 4A) or siGCDH (FIG. 4B) for 72 hours and then treated with 10 μM Cycloheximide (CHX) for indicated time. After quantification, the signals obtained in panel A and B were used to calculate the NRF2/HSP90 ratios and described with respect to CHX. treatment period. FIG. 4C shows immunoprecipitation and Western blot analysis of A375 transfected with indicated constructs. Cells were treated with the proteasomal inhibitor MG132 for 4 hr followed by IP/Western blotting analysis with antibodies to detect K-Glu PTM and NRF2. FIG. 4D shows in vitro glutarylation assay on purified HA-NRF2 following incubation with indicated concentration of glutaryl CoA. FIG. 4E shows in vitro KEAP1 binding analysis performed using purified HA-NRF2 or K-Glu-NRF2 as bait on A375 cell lysates. A representative image of n=3 independent experiments is shown.

FIGS. 10A-10G show that lysine glutarylation increases NRF2 stability and antagonizes KEAP1 binding. FIGS. 10A-10B show Cycloheximide (CHX) chase analysis to measure HA-NRF2 stability in control and (FIG. 10B) GCDH KD and (FIG. 10C) DHTKD1-KD HEK-293T cells ectopically expressing HA-NRF2. HEK293T cells were transfected with indicated constructs for 72 hours and then treated with 10 μM Cycloheximide (CHX) for different time followed by western blotting with indicated antibodies. After quantification, the signals obtained in panel A, B and C were used to calculate the HA-NRF2/HSP90 ratios and described with respect to CHX. treatment period. FIG. 10D shows Immunoprecipitation and Western blot analysis of HA-NRF2 from HEK293T transfected with indicated constructs. HEK 293T cells ectopically expressing HA-NRF2 and treated with the 10 μM proteasomal inhibitor MG132 followed by IP/Western blotting analysis with antibodies to detect K-Glu PTM and HA-NRF2. FIG. 10E shows Enrichment of NRF2 glutarylation in nuclear fraction was measured by HA-NRF2 pull-downs from HEK293T cells transfected with HA-NRF2, after an initial cell fractionation step using MF-membrane fraction; CF-cytoplasmic fraction; NF-nuclear fraction. Successful cell fractionation was confirmed by immunoblotting for specific markers of MF(E-cadherine), CF (GAPDH), and NF (Histone H3). FIG. 10F shows Relative DNA binding activity of purified HANRF2 or K-Glu-NRF2 were measured by in vitro NRF2/ARE EMSA (Electrophoretic-Mobility Shift Assay) using native (non-denaturing) polyacrylamide gels electrophoresis followed by western blotting. A representative image of n=3 independent experiments is shown.

### Example 6: GCDH Inhibition Suppresses Melanoma Growth In Vivo

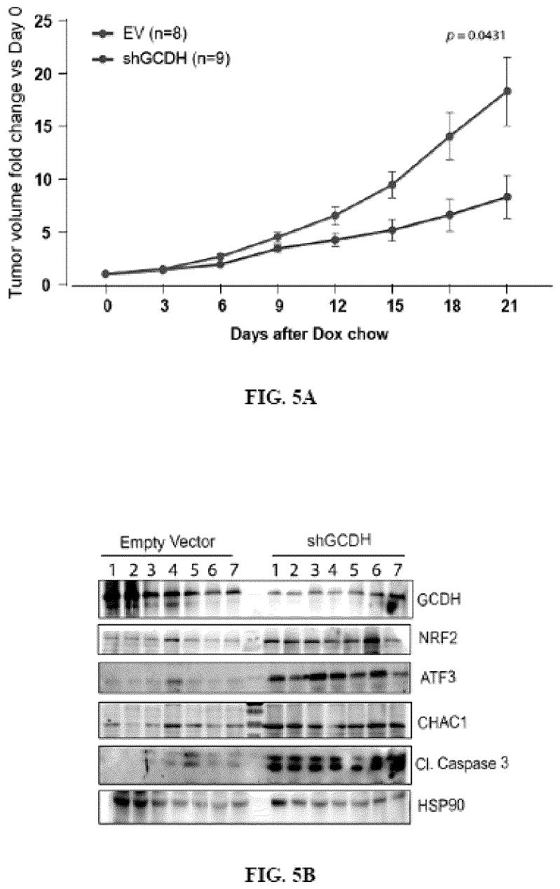

The effects of genetic GCDH inhibition on melanoma growth were monitored in vivo using immunodeficient mice inoculated with human A375 melanoma cells. Genetic inactivation of GCDH was achieved by cloning shRNA targeting GCDH under doxycycline inducible promoter in A375 melanoma cells, that were then inoculated into nude mice. To induce genetic inhibition of GCDH mice were fed with DOX containing chow. Indeed, inhibition of GCDH expression in vivo lead to attenuation of tumor growth, compared to control GCDH expressing tumors (FIG. 5A). Inhibition of GCDH expression and activation of NRF2 and concomitant downstream UPR cell death markers was confirmed for ATF3, CHAC1 and cleave caspase 3 in each of the 7 tumor lysates from GCDH KD compared with control experimental groups (FIG. 53). These finding substantiate the importance of GCDH for melanoma growth; inhibition of GCDH expression results in effective melanoma growth inhibition in vivo.

FIGS. 5A-5B show that GCDH inhibition using inducible shRNA attenuates melanoma proliferation and tumorigenesis. FIG. 5A shows fold change in tumor volume of human melanoma A375 cell line following dox chow treatment. NOD/SCID (NOD.CB17-Prkdcscid/J) mice were injected subcutaneously with 1×106 A375 cells. FIG. 5B shows Western blot analysis present protein levels of GCDH, NRF2, ATF3, CHAC1 and Cl. Caspase 3 in tumor harvested from tumors subjected to control or GCDH KD detailed in panel A. Data are presented as the mean±SEM. Statistical significance (indicated p value relative to control) was calculated using paired t-test.

### Example 7: Discussion

Extensive Efforts have been Made to Identify Tumor Cell Vulnerability to Changes in metabolic signaling. However, tumor cells can activate alternate metabolic pathways to compensate for attenuated metabolic flux. Targeted metabolic pathways can also impair normal cell function or curtails the immune response or other microenvironmental factors that limit tumor growth.

The importance of lysine and tryptophan catabolism for melanoma cells was characterized, and melanoma cell addiction for GCDH signaling was identified. GCDH activity was shown to control NRF2 stability by regulating NRF2 glutarylation. GCDH loss promoted NRF2 glutarylation and increased GCDH stability, promoting melanoma cell death via UPR signaling. Genetic inhibition of GCDH expression was performed, which suppressed melanoma cell growth in culture and tumor growth in vivo.

Melanoma addiction to the mitochondrial protein GCDH was identified, a component in lysine metabolism which controls protein glutarylation. GCDH knockdown promoted apoptotic UPR signaling and cell death in melanoma cells, an activity blocked by knockdown of the upstream lysine catabolism enzyme DHTKD1. Reduced GCDH expression correlated with improved survival of melanoma patients. A key mediator of GCDH-dependent melanoma cell death programs is the transcription factor NRF2, which induces ATF3, CHOP, and CHAC1 transcription linking lysine catabolism with the UPR signaling. NRF2 glutarylation upon GCDH KD increased its stability and DNA binding activity, which coincided with increased transcriptional activity, promoting apoptotic UPR signaling and tumor suppression. In vivo, genetic GCDH inhibition effectively inhibited melanoma tumor growth. The results demonstrate an addiction of melanoma cells to GCDH, which by controlling NRF2 glutarylation limits apoptotic UPR signaling.

Here we establish a novel paradigm and demonstrate melanoma cell addiction to GCDH, one of the enzymes in the multi-step lysine catabolism pathway. Moreover, blocking GCDH activity, but not that of upstream or downstream components of the lysine catabolism pathway, resulted in significant tumor cell death. Our studies define NRF2 as the principal component mediating apoptotic UPR signaling that induces cell death programs. NRF2 glutarylation, seen following GCDH KD, stabilizes NRF2 and likely enhances its transcriptional activation of factors mediating apoptotic UPR signaling. Knockdown of either NRF2, its pro apoptotic transcriptional target ATF337 or GCDH-upstream enzyme DTHKD1 effectively blocked cell death phenotypes seen upon GCDH KD, indicating that a pathway controlled by GCDH activity allows survival of melanoma cells.

NRF2 reportedly exhibits both oncogenic and tumor suppressor activities, in different cancer models, although mechanisms determining those activities are not well understood. In melanoma, NRF2 has been previously shown to affect innate immune responses and oxidative stress. Additionally, high levels of NRF2 protein were found to be associated with a poor prognosis in melanoma irrespective of oxidative stress. Our findings show that NRF2 exhibits tumor suppressor activity upon glutarylation and suggest that NRF2 glutarylation induced by GCDH loss-of-function both promotes its dissociation from the E3 ubiquitin ligase KEAP1 and enhances it stability, which then increases NRF2-dependent expression of select gene set that mediate apoptotic UPR signaling. NRF2 glutarylation occurs on lysines that may otherwise serve as ubiquitin acceptor sites, reducing its ubiquitination and enhancing its stability. Mapping NRF2 lysine glutarylation site(s) would be desirable; however, similar to lysine ubiquitination, glutarylation may be promiscuous, such that when some sites are unavailable, others are modified. Relevant to transcriptional effects, we perform in vitro gel shift assays and showed that NRF2 glutarylation enhances its binding to the known NRF2 response element. However, it is also possible that glutarylated NRF2 possesses greater affinity to form complexes with transcriptional co-activators or co-suppressors, or with epigenetic regulators governing translation initiation complex assembly, each of which would define a select transcriptional readout.

Our findings demonstrate that addiction to GCDH signaling is observed in melanoma cells but not in breast, colon or prostate tumor cells. Important support for these findings comes from clinical data, in which low GCDH expression coincided with better patient outcomes in melanoma but not in other tumor types. One explanation for selective GCDH dependency is the neural crest origin of melanoma, resembling phenotypes seen in brain of the GCDH KO mice. Equally plausible is that different proteins undergo glutarylation following GCDH loss in tumors other than melanoma, which may alter different signaling pathways. In support of this possibility is the observation of numerous glutarylated proteins upon GCDH KDin different tissues, which could modify activity of distinct drivers of oncogenesis depending on the substrate.

Would targeting GCDH offer a novel therapeutic modality for melanoma? Data from total KO mice suggest that ablation of either GCDH or other components of the lysine catabolism pathway 31, 32 does not have a major impact on either normal development or tissue homeostasis, and mice are viable with minor deficiencies. However, mice globally deficient in GCDH acquire vulnerability to excessive lysine or high protein diets 33, implying that a ketogenic diet may enhance cell death in GCDH-low tumor cells, a possibility deserving further assessment. Our in vivo data supports effectiveness of genetic GCDH inhibition, which attenuated melanoma growth in immunodeficient mice, suggesting that GCDH may be required for tumor cell growth in vivo. Further work is required to examine the effect of melanoma addiction to GCDH on the TME including anti-tumor immunity. Melanoma addiction to GCDH well illustrates the selective advantage of select metabolic cue for distinct tumor types.

What is claimed is:

1. A method of treating a condition, the method comprising administering to a subject in need thereof a therapeutically-effective amount of a compound, wherein the compound modulates a glutaryl-CoA dehydrogenase (GCDH) protein activity.

2. The method of claim 1, wherein the administering decreases GCDH protein activity in the subject.

3. The method of claim 1, wherein the administering decreases a GCDH protein expression level in the subject.

4. The method of claim 1, wherein the administering decreases GCDH protein enzyme activity in the subject.

5. The method of claim 1, wherein the compound comprises a nucleic acid.

6. The method of claim 1, wherein the compound comprises a ribonucleic acid.

7. The method of claim 6, wherein the ribonucleic acid comprises a microRNA.

8. The method of claim 6, wherein the ribonucleic acid comprises a short hairpin RNA.

9. The method of claim 6, wherein the ribonucleic acid comprises a small interfering RNA.

10. The method of claim 6, wherein the ribonucleic acid comprises a sequence that is at least about 80% identical to SEQ ID NO: 13.

11. The method of claim 6, wherein the ribonucleic acid comprises a sequence that is at least about 80% identical to SEQ ID NO: 14.

12. The method of claim 6, wherein the ribonucleic acid comprises a sequence that is at least about 90% identical to SEQ ID NO: 13.

13. The method of claim 6, wherein the ribonucleic acid comprises a sequence that is at least about 90% identical to SEQ ID NO: 14.

14. The method of claim 6, wherein the ribonucleic acid comprises a sequence that is about 100% identical to SEQ ID NO: 13.

15. The method of claim 6, wherein the ribonucleic acid comprises a sequence that is about 100% identical to SEQ ID NO: 14.

16. The method of claim 1, wherein the condition is a cancer.

17. The method of claim 16, wherein the cancer is a glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, skin cancer, or blood cancer.

18. The method of claim 16, wherein the cancer is a skin cancer.

19. The method of any one of claim 16, wherein the cancer is a basal cell carcinoma.

20. The method of any one of claim 16, wherein the cancer is a squamous cell carcinoma or a melanoma.
